(19) United States
(12) Patent Application Publication
(10) Pub. No.: US 2003/0105379 A1
(43) Pub. Date: Jun. 5, 2003

(54) Treatment of adsorbent to enhance adsorbent capacity for acetylenic compounds

(76) Inventors: Brian C. Bahr, Batavia, IL (US); Michael J. Foral, Aurora, IL (US); Mark P. Kaminsky, Friendswood, TX (US); Diadema N. Ventura, Lisle, IL (US); Tom Wairegi, Houston, TX (US)

Correspondence Address: CAROL WILSON, BP AMERICA INC., MAIL CODE 5 EAST, 4101 WINFIELD ROAD, WARRENVILLE, IL, 60555, US

(21) Appl. No.: 09/997,903

(22) Filed: Nov. 30, 2001

(51) Int. Cl. C07C 7/12; C07C 7/00

(52) U.S. Cl. 585/820; 585/829; 585/826; 585/809

(57) ABSTRACT

Processes using heterogeneous adsorbents are disclosed for purification of olefin streams, such as are produced by thermal cracking of hydrocarbons, to obtain a feedstock suitable for formation of olefin polymers. These purification processes comprises: providing an impure gaseous mixture; passing the impure mixture through a bed of regenerated adsorbent which is free of a substantial amount of carbon monoxide; effecting, in the presence of an essentially dihydrogen-free atmosphere within the bed, selective adsorption of the contained acetylenic impurities with the adsorbent until levels of the acetylenic impurities in the effluent mixture increase to a limiting level in a range downward from about 1 parts per million by volume; and thereafter regenerating the resulting bed of adsorbent in the presence of a reducing gas comprising dihydrogen which reducing gas is free of a substantial amount of carbon monoxide.

### FIELD OF THE INVENTION

The field of this invention relates to use of heterogeneous adsorbents in purification of relatively impure olefins to obtain a feedstock suitable for formation of olefin polymers. More particularly, this invention concerns purification by passing an olefinic process stream, containing small amounts of acetylenic impurities, carbon oxides and/or other organic components which are, typically, impurities in cracked gas, through a bed of regenerated adsorbent which is free of a substantial amount of carbon monoxide; effecting, in the presence of an essentially dihydrogen-free atmosphere within the bed, selective adsorption of the contained acetylenic impurities with the adsorbent until levels of the acetylenic impurities in the effluent mixture increase to a limiting level in a range downward from about 1 parts per million by volume; and thereafter regenerating the resulting bed of adsorbent in the presence of a reducing gas comprising dihydrogen which reducing gas is free of a substantial amount of carbon monoxide.

Processes according to this invention are particularly useful where the olefin being purified is ethylene and/or propylene formed by thermal cracking of hydrocarbon feedstocks.

### BACKGROUND OF THE INVENTION

As is well known, olefins, or alkenes, are a homologous series of hydrocarbon compounds characterized by having a double bond of four shared electrons between two carbon atoms. The simplest member of the series, ethylene, is the largest volume organic chemical produced today. Olefins including, importantly, ethylene, propylene and smaller amounts of butadiene, are converted to a multitude of intermediate and end products on a large scale, mainly polymeric materials.

Commercial production of olefins is, almost exclusively, accomplished by pyrolysis of hydrocarbons in tubular reactor coils installed in externally fired heaters. Thermal cracking feed stocks include streams of ethane, propane or a hydrocarbon liquid ranging in boiling point from light straight-run gasoline through gas oil. Because of the very high temperatures employed, commercial olefin processes invariably coproduce significant amounts of acetylene and methyl acetylene. Required separation of the acetylene from the primary olefin can, considerably, increase the plant cost.

In a typical ethylene plant the cracking section represents about 25 percent of the cost of the unit while the compression, heating, dehydration, recovery and refrigeration sections represent the remaining about 75 percent of the total. This endothermic process is carried out in large pyrolysis furnaces with the expenditure of large quantities of heat which is provided in part by burning the methane produced in the cracking process. After cracking, the reactor effluent is put through a series of separation steps involving cryogenic separation of products such as ethylene and propylene. The total energy requirements for the process are thus very large and ways to reduce it are of substantial commercial interest. In addition, it is of interest to reduce the amount of methane and heavy fuel oils produced in the cracking processor to utilize it other than for its fuel value.

Hydrocarbon cracking is carried out using a feed which is ethane, propane or a hydrocarbon liquid ranging in boiling point from light straight-run gasoline through gas oil. Ethane, propane, liquid naphthas, or mixtures thereof are preferred feed to a hydrocarbon cracking unit. Hydrocarbon cracking is, generally, carried out thermally in the presence of dilution steam in large cracking furnaces which are heated by burning, at least in part, methane and other waste gases from the olefins process resulting in large amounts of NOx pollutants. The hydrocarbon cracking process is very endothermic and requires large quantities of heat per pound of product. However, newer methods of processing hydrocarbons utilizes at least to some extent catalytic processes which are better able to be tuned to produce a particular product slate. The amount of steam used per pound of feed in the thermal process depends to some extent on the feed used and the product slate desired. Typically, steam pressures are in the range of about 30 lbs per sq in to about 80 lbs per sq in, and amounts of steam used are in the range of about 0.2 pounds of steam per pound of feed to 0.7 pounds of per pound of feed. The temperature, pressure and space velocity ranges used in thermal hydrocarbon cracking processes to some extent depend upon the feed used and the product slate desired which are well known as may be appreciated by one skilled in the art.

The type of furnace used in the thermal cracking process is also well known. However the ceramic honeycomb furnace which is described in U.S. Pat. No. 4,926,001, the contents of which patent are specifically incorporated herein by reference, is an example of a new type of cracking which could have a special utility for this process.

Several methods are known for separation of an organic gas containing unsaturated linkages from gaseous mixtures. These include, for instance, cryogenic distillation, liquid adsorption, membrane separation and the so called “pressure swing adsorption” in which adsorption occurs at a higher pressure than the pressure at which the adsorbent is regenerated. Cryogenic distillation and liquid adsorption are common techniques for separation of carbon monoxide and alkenes from gaseous mixtures containing molecules of similar size, e.g., nitrogen or methane. However, both techniques have disadvantages such as high capital cost and high operating expenses. For example, liquid adsorption techniques suffer from solvent loss and need a complex solvent make-up and recovery system.

Molecular sieves which selectively adsorb carbon monoxide from gaseous mixtures by chemisorption are also known. U.S. Pat. No. 4,019,879 and U.S. Pat. No. 4,034,065 refer to use of high silica zeolites, which have relatively high selectivities for carbon monoxide, in the pressure swing adsorption method. However, these zeolites only have moderate capacity for carbon monoxide and more particularly require very low vacuum pressures to recover the adsorbed gases and/or to regenerate the zeolite.

U.S. Pat. No. 4,717,398 describes a pressure swing adsorption process for selective adsorption and subsequent recovery of an organic gas containing unsaturated linkages from gaseous mixtures by passing the mixture over a zeolite ion-exchanged with cuprous ions (Cu I) characterized in that the zeolite has a faujasite type crystalline structure (Y).

Kokai JP Number 50929-1968 describes a method of purifying vinyl compounds containing up to about 10 percent by weight of acetylene compounds including ethyl acetylene, vinyl acetylene and phenyl acetylene whereby the acetylene compounds are adsorbed in an adsorption agent of 1-valent and/or 0-valent copper and/or silver supported on inert carrier such as δ-alumina, silica or active carbon. However, it is well known that acetylene and these acetylene compounds react with copper and/or silver to from copper acetylide or silver acetylide. Both the acetylide of copper and silver are unstable compounds. Because they are explosive under some conditions their possible formation presents safety problems in operation and in handling adsorbent containing such precipitates.

More recently German Disclosure Document 2059794 describes a liquid adsorption process for purification of paraffinic, olefinic and/or aromatic hydrocarbons with an adsorption agent consisting in essence of a complex of a copper (Cu I)-salt with an alkanolamine such as monoethanolamine, monoisopropanolamine, diethanolamine, triethanolamine and arylalkanolmines, and optionally in the presence of a glycol or polyglycol. However, the product stream is contaminated with unacceptable levels of components of the such agents absorbed in the hydrocarbon flow. While such contamination might be removable using an additional bed of silica gel, aluminum oxide or a wide-pored molecular sieve, this would involve additional capital costs, operation expenses and perhaps safety problems.

Processes using heterogeneous adsorbents are known for purification of olefins, such as are typically produced by thermal cracking of suitable hydrocarbon feedstocks, by passing a stream of olefin through a particulate bed of support material on which is dispersed a metallic element. More recently U.S. Pat. No. 6,080,905 and U.S. Pat. No. 6,124,517 in the name of Mark P. Kaminsky, Shiyou Pei, Richard A Wilsak, and Robert E. Whittaker describe adsorption which is carried out in an essentially dihydrogen-free atmosphere within the bed. Adsorption of the contained acetylenic impurities is continued until levels of acetylenic impurities in the effluent stream increase to a predetermined level. Thereafter the resulting bed of adsorbent is regenerated using hydrogen to effect release of the contained acetylenic impurities from the adsorbent. However, there remains a need to increase the capacity of adsorbents for acetylenics whereby the useful life of the adsorbent bed between regenerations is increased.

Olefin-paraffin separations represent a class of most important and also most costly separations in the chemical and petrochemical industry. Cryogenic distillation has been used for over 60 years for these separations. They remain to be the most energy-intensive distillations because of the close relative volatilities. For example, ethane-ethylene separation is carried out at about −25° C. and 320 pounds per square inch gauge pressure (psig) in a column containing over 100 trays, and propane-propylene separation is performed by an equally energy-intensive distillation at about −30° C. and 30 psig.

Impurity refers to compounds that are present in the olefin plant feedstocks and products. Well-defined target levels exist for impurities. Common impurities in ethylene and propylene include: acetylene, methyl acetylene, methane, ethane, propane, propadiene, and carbon dioxide. Listed below are the mole weight and atmospheric boiling points for the light products from thermal cracking and some common compounds potentially found in an olefins unit. Included are some compounds which have similar boiling temperatures to cracked products and may be present in feedstocks or produced in trace amounts during thermal cracking.

| | Mole | Normal Boiling |
|---|---|---|
| Compound | Weight | Point, ° C. |
| Hydrogen | 2.016 | −252.8 |
| Nitrogen | 28.013 | −195.8 |
| Carbon monoxide | 28.010 | −191.5 |
| Oxygen | 31.999 | −183.0 |
| Methane | 16.043 | −161.5 |
| Ethylene | 28.054 | −103.8 |
| Ethane | 30.070 | −88.7 |
| Phosphine | 33.970 | −87.4 |
| Acetylene* | 26.038 | −84.0 |
| Carbon dioxide* | 44.010 | −78.5 |
| Radon | 222.00 | −61.8 |
| Hydrogen sulfide | 34.080 | −60.4 |
| Arsine | 77.910 | −55.0 |
| Carbonyl sulfide | 60.070 | −50.3 |
| Propylene | 42.081 | −47.8 |
| Propane | 44.097 | −42.1 |
| Propadiene (PD) | 40.065 | −34.5 |
| Cyclo-propane | 42.081 | −32.8 |
| Methyl acetylene | 40.065 | −23.2 |
| Water | 18.015 | 100. |

*Sublimation temperature

Recently the trend in the hydrocarbon processing industry is to reduce commercially acceptable levels of impurities in major olefin product streams, i.e., ethylene, propylene, and hydrogen. Need for purity improvements is directly related to increasing use of higher activity catalysts for production of polyethylene and polypropylene, and to a limited extent other olefin derivatives.

It is known that acetylenic impurities can be selectively hydrogenated and thereby removed from such product streams by passing the product stream over an acetylene hydrogenation catalyst in the presence of dihydrogen (molecular hydrogen, H₂). However, these hydrogenation processes typically result in the deposition of carbonaceous residues or “green oil” on the catalyst which deactivates the catalyst. Therefore, acetylene hydrogenation processes for treating liquid or liquefiable olefins and diolefins typically include an oxygenation step or a “burn” step to remove the deactivating carbonaceous residues from the catalyst followed by a hydrogen reduction step to reactivate the hydrogenation catalyst. For example, see U.S. Pat. No. 3,755,488 to Johnson et al., U.S. Pat. No. 3,792,981 to Hettick et al., U.S. Pat. No. 3,812,057 to Morgan and U.S. Pat. No. 4,425,255 to Toyoda. However, U.S. Pat. No. 3,912,789 and U.S. Pat. No. 5,332,705 state that by using selected hydrogenation catalysts containing palladium, at least partial regeneration can be accomplished using a hydrogenation step alone at high temperatures (600° F.-700° F.) and in the absence of an oxygenation step.

Selective hydrogenation of the about 2000 to 4000 parts per million of acetylenic impurities to ethylene is, generally, a crucial operation for purification of olefins produced by thermal steam cracking. Typical of a small class of commercially useful catalysts are materials containing very low levels of an active metal supported on an inert carrier, for example a particulate bed having less than about 0.03 percent (300 ppm) palladium supported on the surface skin of carrier pellets having surface area of less than about 10 m²/gm.

Many commercial olefin plants using steam crackers use, generally, front-end acetylene converters, i.e., the hydrogenation unit is fed C₃ and lighter cracked gas which feed has a high enough concentration of hydrogen to easily hydrogenate the acetylenic impurities, however, when run improperly, will also hydrogenate a large fraction of the ethylene and propylene product. Both hydrogenation of acetylene and ethylene are highly exothermic as shown below:

C₂H₂+H₂ - - - >C₂H₄ H=−41 kcal/mole

C₂H₄+H₂ - - - >C₂H₆ H=−32.7 kcal/mole

Accelerated catalyst deactivation and thermal runaways caused by loss in catalyst selectivity are common problems which plague acetylene converters. Such problems result in unscheduled shutdowns and increased costs to replace deactivated catalyst.

The problem of over-hydrogenation is aggravated because the rate constant for ethylene hydrogenation to ethane is 100 times faster than for the hydrogenation of acetylene to ethylene. As a means to avoid a C₂H₄ hydrogenation thermal runaway, acetylene, carbon monoxide and diolefins concentrations must, therefore, be high enough to cover most of the active sites so that none are left to adsorb ethylene. For example, acetylene, carbon monoxide, methyl acetylene, and propadiene have bond strengths to palladium which are stronger than the ethylene to palladium bonds. Selection of active metal, size of the metal particles and other physical and chemical factors ultimately affect the “operating temperature window” which is the delta of temperature between acetylene conversion to ethylene (typically in a range from about 100° F. to about 150° F.) and thermal runaway where all molecular hydrogen is converted and a large amount of the ethylene is converted to ethane (about 170° F. to about 225° F.). The wider the window, the safer is operation of the unit.

It is therefore a general object of the present invention to provide an improved process which overcomes the aforesaid problem of prior art methods, for production of olefins from thermal cracking of hydrocarbon feed stocks which olefin can be used for manufacture of polymeric materials using higher activity catalysts.

More particularly, it is an object of the present invention to provide an improved method for purification of ethylene and/or propylene containing small amounts of acetylenic impurities, carbon oxides and/or other organic components that are impurities in olefinic process streams, by passing the impure olefin stream through a particulate bed of heterogeneous adsorbent comprising a metal supported on a high surface area carrier, under conditions suitable for reversible adsorption of alkyne impurities.

It is another object of the present invention to provide an improved aforesaid purification method that employs an adsorbent that, even after a substantial period of aging, exhibits ability to withstand repeated regenerations and yet retain useful adsorption capacity.

It is further an object of this invention to provide an improved process for regeneration of adsorbent loaded with acetylenic impurities.

Other objects and advantages of the invention will become apparent upon reading the following detailed description and appended claims.

### SUMMARY OF THE INVENTION

Economical processes are disclosed for purification of a relatively impure olefins such as is, for example, produced by thermal cracking of hydrocarbons. More particularly, this invention is a process for purification of an olefin stream to obtain a feedstock suitable for formation of olefin polymers. In one aspect integrated processes of the invention comprise: providing an impure gaseous mixture comprising at least one olefin of from 2 to about 8 carbon atoms, acetylenic impurities having the same or similar carbon content in an amount of up to about 1 percent by volume based upon the total amount of olefin present and optionally saturated hydrocarbon gases; passing the impure mixture through a bed of regenerated adsorbent which is free of a substantial amount of carbon monoxide, the adsorbent comprising predominantly a support material having high surface area on which is dispersed at least one metallic element in the zero valent state selected from the group consisting of chromium, iron, cobalt, nickel, copper, ruthenium, palladium, silver and platinum, to effect, under conditions suitable for adsorption within the bed, selective adsorption and/or complexing of the contained acetylenic contaminants with the adsorbent, and thereby obtain an effluent mixture which contains less than about 1 part per million by volume of the acetylenic impurities and an amount of carbon monoxide less than is deleterious to formation of olefin polymers; and thereafter regenerating the resulting bed of adsorbent in the presence of a reducing gas comprising dihydrogen which is free of a substantial amount of carbon monoxide, to effect release of the contained acetylenic impurities from the adsorbent. Advantageously, integrated processes of the invention provide purified product containing less than about 1 parts per million by weight, and frequently even less than about 0.5 parts per million by weight.

An aspect of special significance is the separation of acetylenic impurities from ethylene or propylene containing small amounts of acetylene, i.e., less than about 5000 parts per million by weight of one or more acetylenic impurities. Where the olefin in the gaseous mixture being purified is predominantly ethylene or propylene, the gaseous mixture beneficially contains less than about 0.5 parts per million by volume of dihydrogen and less than about 1 parts per million by volume of mercury-containing, arsenic-containing, and sulfur-containing components.

Another aspect of special significance is conducting the separation of acetylenic impurities from the gaseous mixture by passing the gaseous mixture through the bed of adsorbent at temperatures in a range downward from about 20° C., preferably at temperatures in a range downward from about 20° C. to about negative 35° C., and more preferably at temperatures in a range of from about negative 35° C. to about 5° C. for best results.

In yet another aspect the invention is a process for purification of olefins, such as are produced by thermal cracking of hydrocarbons, to obtain a feedstock suitable for formation of olefin polymers, which purification process comprises: providing an impure gaseous mixture; passing the impure mixture through a bed of regenerated adsorbent which is free of a substantial amount of carbon monoxide; effecting, in the presence of an essentially dihydrogen-free atmosphere within the bed, selective adsorption of the contained acetylenic impurities with the adsorbent until levels of the acetylenic impurities in the effluent mixture increase to a limiting level in a range downward from about 1 parts per million by volume; and thereafter regenerating the resulting bed of adsorbent in the presence of a reducing gas comprising dihydrogen which reducing gas is free of a substantial amount of carbon monoxide. The impure gaseous mixture comprises at least about 99 percent by volume of an olefin having from 2 to about 4 carbon atoms, and acetylenic impurities having the same or similar carbon content in an amount in a range upward from about 1 to about 1000 parts per million by volume based upon the total amount of olefin present and optionally saturated hydrocarbon gases. The impure mixture is passed through a bed of regenerated adsorbent which is free of a substantial amount of carbon monoxide. Advantageously, the adsorbent comprises predominantly a support material selected from the group alumina, silica, active carbon, clay and zeolites having surface area in a range of from about 10 to about 2,000 square meters per gram as measured by the BET gas adsorption method, on which is dispersed at least one metallic element in the zero valent state selected from the group consisting of chromium, iron, cobalt, nickel, copper, ruthenium, palladium, silver and platinum, to effect, under conditions suitable for adsorption within the bed, selective adsorption and/or complexing of the contained acetylenic contaminants with the adsorbent. In the presence of an essentially dihydrogen-free atmosphere within the bed, selective adsorption and/or complexing of the contained acetylenic impurities with the adsorbent is effected, until levels of the acetylenic impurities in the effluent mixture increase to a limiting level in a range downward from about 1 parts per million by volume. Thereafter regenerating the resulting bed of adsorbent in the presence of a reducing gas comprising dihydrogen which reducing gas is free of a substantial amount of carbon monoxide, to effect release of the contained acetylenic impurities from the adsorbent.

In yet another aspect of special significance is a process for purification of a predominantly ethylene stream to obtain a feedstock suitable for formation of polymers thereof. In this aspect the purification process comprises: providing an impure gaseous stream comprising at least about 90 percent by volume of ethylene, and acetylene in an amount in a range upward from about 1 to about 1000 parts per million by volume based upon the total amount of ethylene present and optionally saturated hydrocarbon gases; passing the impure mixture through a bed of adsorbent which is free of a substantial amount of carbon monoxide, the adsorbent comprising at least about 90 weight percent of gamma alumina having surface area in a range of from about 150 to about 350 square meters per gram as measured by the BET gas adsorption method, on which is dispersed is at least one element selected from the group consisting of iron, cobalt, nickel, copper, palladium, silver and platinum, in the zero valent state, to effect, under conditions suitable for adsorption within the bed, selective adsorption and/or complexing of the contained acetylene contaminant with the adsorbent, thereby obtain an effluent stream of feedstock which contains less than about 0.5 parts per million by volume of carbon monoxide and less than about 1 parts per million by volume of the acetylene and; effecting, in the presence of an essentially dihydrogen-free atmosphere within the bed, selective adsorption and/or complexing of the contained acetylene with the adsorbent, until levels of the acetylene in the effluent stream increase to a limiting level in a range downward from about 1 parts per million by volume; and thereafter regenerating the resulting bed of adsorbent in the presence of a reducing gas comprising dihydrogen which reducing gas is free of a substantial amount of carbon monoxide, to effect release of the contained acetylene from the adsorbent.

Beneficially, the adsorbent comprises at least about 90 weight percent of a gamma alumina having surface area in a range of from about 150 to about 350 square meters per gram as measured by the BET gas adsorption method, the metal dispersed on the support material is palladium, and the absorbent has a palladium content in a range of from about 0.01 to about 10 percent based on the total weight of the adsorbent, and wherein the gaseous mixture, while passing through the bed, is at temperatures in a range of from about negative 35° C. to about 5° C.

A preferred class of adsorbents useful in processes according the invention, comprises at least about 90 weight percent of a gamma alumina having surface area in a range of from about 80 to about 500 square meters per gram as measured by the BET gas adsorption method, and contains less than 500 parts per million by weight of a sulfur-containing component, calculated as elemental sulfur. More preferred are the adsorbent which comprises at least about 90 weight percent of a gamma alumina having surface area in a range of from about 150 to about 350 square meters per gram as measured by the BET gas adsorption method, and wherein the metal dispersed on the support material is palladium, and the absorbent has a palladium content in a range of from about 0.01 to about 10 percent based on the total weight of the adsorbent.

For a more complete understanding of the present invention, reference should now be made to the embodiments described below by way of examples of the invention.

### BRIEF DESCRIPTION OF THE INVENTION

Processes of this invention are particularly suitable for use in purification of aliphatically unsaturated organic compounds produced, generally, by thermal cracking of hydrocarbons.

Aliphatically unsaturated compounds of most interest with regard to purification by the method of the present invention, have two to about eight carbon atoms, preferably two to about four carbon atoms, and more preferably ethylene or propylene. The separation of acetylenic impurities from ethylene or propylene which may be contained in admixtures with other normally gaseous materials, such as one or more of ethane, methane, propane and oxides of carbon is of particular importance. For example mixtures serving as a source of ethylene containing feed for the process may contain about 1 to about 99 weight percent ethylene, about 0 to about 50 weight percent ethane and/or about 0 to about 50 weight percent methane.

Generally acetylenic impurities described in this invention are expressed by the formula

R—C≡CH

where R is hydrogen or a hydrocarbon group of up to 6 carbon atoms.

It is desirable to treat the gaseous mixture used in the process of the present invention to remove any gaseous hydrogen and or carbon monoxide. The amount of hydrogen in the gaseous mixture should suitably be reduced to below 10 parts per million by weight, preferably below 2 parts per million by weight and most preferably below 1 parts per million by weight, prior to contact with the adsorbent.

Similarly, any mercury-containing, arsenic-containing, and sulfur-containing components, e.g., hydrogen sulfide, present in the gaseous mixture fed to the particulate bed of adsorbent should suitably be removed therefrom in any known manner in order to avoid the risk of poisoning the dispersed metal. The hydrocarbon mixture used in the process of the present invention is suitably a cracked gas from which the majority of the C₅ and higher hydrocarbons have been removed. The gaseous mixture may thus comprise ethylene, propylene, butenes, methane, ethane, propane and butane. Small amounts of pentanes and pentenes can be tolerated in the gaseous mixture.

In preferred embodiments of processes according to the invention, the olefin in the gaseous mixture being purified is predominantly ethylene or propylene, the gaseous mixture contains less than about 0.5 parts per million by volume of hydrogen and less than about 1 parts per million by volume of mercury-containing, arsenic-containing, and sulfur-containing components, each calculated as the element, and wherein the gaseous mixture, while passing through the bed, is at temperatures in a range upward from about −78° C. to about 100° C., preferably in a range of from about −35° C. to about 65° C., and more preferably in a range of from about −10° C. to about 55° C.

The gaseous mixture used in the process of the present invention may also comprise water and may optionally be saturated with water.

Broadly, according to the present invention, there is provided a particulate bed of adsorbent comprising predominantly a support material having high surface area on which is dispersed at least one metallic element selected from the group consisting of chromium, iron, cobalt, nickel, copper, ruthenium, palladium, silver and platinum, and preferably at least one metallic element selected from the group consisting of iron, cobalt, nickel, copper, palladium, silver and platinum. Suitable adsorbents exhibit, in the presence of an essentially dihydrogen-free atmosphere within the bed, selective and reversible adsorption and/or complexing of the acetylenic impurities with the adsorbent. According to the present invention dispersed metal content is in a range of from about 0.01 to about 40 percent based on the total weight of the adsorbent. Preferably dispersed metal content is in a range of from about 0.01 to about 20 percent based on the total weight of the adsorbent.

The adsorbent can, optionally, further comprise one or more elements selected from the group consisting of lithium, sodium, potassium, zinc, molybdenum, tin, tungsten, and iridium, dispersed on the support material. Preferably the adsorbent further comprises a member selected from the group consisting of lithium, sodium, potassium, zinc, molybdenum, and tin dispersed on the support material.

For processes according to invention the metal dispersed on the support material is, advantageously, at least one element selected from the group consisting of iron, cobalt, nickel, copper, palladium, silver and platinum, and the absorbent has a dispersed metal content in a range of from about 0.05 to about 20 percent based on the total weight of the adsorbent.

Another class adsorbents useful for processes according to invention comprises a dispersion of copper or silver and one metallic element selected from the group consisting of chromium, iron, cobalt, nickel, ruthenium, palladium, and platinum, preferably palladium.

More preferred for processes according to this invention are adsorbents having palladium metal dispersed on the support, and the absorbent has a palladium content in a range of from about 0.05 to about 10 percent, more preferred palladium content in a range of from about 0.1 to about 5.0 percent based on the total weight of the adsorbent.

High metal dispersion and loading resulted in higher metal surface area. Capacity of an adsorbent is, typically, related directly to metal surface area. Any method which increases and/or maintains high metal surface area is, therefore, beneficial to achieving high acetylene adsorption capacity.

Preferred for processes according to this invention are adsorbents having a dispersion value of at least about 10 percent, preferably in a range upward from about 20 percent to about 80 percent. Dispersion is a measure of the accessibility of the active metals on the adsorbent. Such dispersion methods are discussed in H. C. Gruber's, Analytical Chemistry, Vol. 13, p. 1828, (1962). The absorbents for use in this invention were analyzed for dispersion using a pulsed carbon monoxide technique as described in more detail in the Examples. Palladium containing adsorbents having large dispersion values are desired because more of the palladium metal is available for reaction.

Support materials are, advantageously, selected from the group consisting of alumina, silica, carbon, clay and zeolites (molecular sieves). Surface areas of support materials are, preferably, in a range of from about 10 to about 2,000 square meters per gram as measured by the Bet gas adsorption method.

A preferred class of active carbons useful herein are materials disclosed in commonly assigned U.S. Pat. No. 4,082,694 to Arnold N. Wennerberg and Thomas M. O'Grady, which patent is incorporated herein by reference. Such suitable active carbon products are produced from carbonaceous material by a staged temperature process which provides improved yield and processability during manufacture. A source of carbonaceous material, such as crushed coal, coal coke, petroleum coke or a mixture thereof, is heated with agitation in the presence of a substantial weight ratio of potassium hydroxide at a first lower temperature to dehydrate the combination. Thereafter the temperature is raised to a second higher temperature to activate the combination which is thereafter cooled and washed to remove inorganic matter and form a high surface area active carbon having a cage-like structure exhibiting micro-porosity, good bulk density and Total Organic Carbon Index.

Active carbon products for use as supports according to this invention have, preferably, an effective surface area greater than about 2,300 square meters per gram and, more preferably, greater than about 2,700 square meters per gram and, most preferably, above about 3,000 square meters per gram as measured by the BET method. Active carbon products for use as supports have, typically, a bulk density greater than about twenty-five hundredths grams per cubic centimeter and, preferably greater than about twenty-seven hundredths grams per cubic centimeter and, more preferably, above about three-tenths gram per cubic centimeter. Further, useful active carbon products preferably have a Total Organic Carbon Index greater than about 300, more preferably, greater than about 500 and, most preferably, greater than about 700.

Generally, the term “molecular sieve” includes a wide variety of positive-ion-containing crystalline materials of both natural and synthetic varieties. They are generally characterized as crystalline aluminosilicates, although other crystalline materials are included in the broad definition. The crystalline aluminosilicates are made up of networks of tetrahedra of SiO₄ and AlO₄ moieties in which the silicon and aluminum atoms are cross-linked by the sharing of oxygen atoms. The electrovalence of the aluminum atom is balanced by the use of positive ions, for example, alkali-metal or alkaline-earth-metal cations.

Zeolitic materials, both natural and synthetic, useful herein have been demonstrated in the past to have catalytic capabilities for many hydrocarbon processes. Zeolitic materials, often referred to as molecular sieves, are ordered porous crystalline aluminosilicates having a definite structure with large and small cavities interconnected by channels. The cavities and channels throughout the crystalline material are generally uniform in size allowing selective separation of hydrocarbons. Consequently, these materials in many instances have come to be classified in the art as molecular sieves and are utilized, in addition to the selective adsorptive processes, for certain catalytic properties. The catalytic properties of these materials are also affected, to some extent, by the size of the molecules which are allowed selectively to penetrate the crystal structure, presumably to be contacted with active catalytic sites within the ordered structure of these materials.

In the past various molecular sieve compositions natural and synthetic have been found to be useful for a number of hydrocarbon conversion reactions. Among these are alkylation, aromatization, dehydrogenation and isomerization. Among the sieves which have been used are Type A, X, Y and those of the MFI crystal structure, as shown in “Atlas of Zeolite Structure Types,” Second Revised Edition 1987, published on behalf of the Structure Commission of the International Zeolite Associates and incorporated by reference herein. Representative of the last group are ZSM-5 and AMS borosilicate molecular sieves.

Prior art developments have resulted in the formation of many synthetic crystalline materials. Crystalline aluminosilicates are the most prevalent and, as described in the patent literature and in the published journals, are designated by letters or other convenient symbols. Exemplary of these materials are Zeolite A (Milton, in U.S. Pat. No. 2,882,243), Zeolite X (Milton, in U.S. Pat. No. 2,882,244), Zeolite Y (Breck, in U.S. Pat. No. 3,130,007), Zeolite ZSM-5 (Argauer, et al., in U.S. Pat. No. 3,702,886), Zeolite ZSM-11 (Chu, in U.S. Pat. No. 3,709,979), Zeolite ZSM-12 (Rosinski, et al., in U.S. Pat. No. 3,832,449), and others.

Manufacture of the ZSM materials utilizes a mixed base system in which sodium aluminate and a silicon containing material are mixed together with sodium hydroxide and an organic base, such as tetrapropylammonium hydroxide and tetrapropylammonium bromide, under specified reaction conditions, to form the crystalline aluminosilicate, preferably a crystalline metallosilicate exhibiting the MFI crystal structure.

A preferred class of molecular sieves useful, according to the present invention, are crystalline borosilicate molecular sieves disclosed in commonly assigned U.S. Pat. Nos. 4,268,420, 4,269,813, 4,292,457, and 4,292,458 to Marvin R. Klotz, which are incorporated herein by reference.

### BRIEF DESCRIPTION OF PREFERRED EMBODIMENTS OF THE INVENTION

While this invention is susceptible of embodiment in many different forms, this specification discloses only some specific forms as an example of the use of the invention. In particular, preferred embodiments of the invention for purification of a gaseous mixture comprising olefin preferably an olefin of from two to about eight carbon atoms having a single double bond, acetylenic impurities having the same or similar carbon content and optionally alkanes (paraffin hydrocarbons) and/or alkenes having more than one double bond (di- or tri-olefin hydrocarbons) produced by thermal cracking of hydrocarbons are illustrated and described. The invention is not intended to be limited to the embodiments so described, and the scope of the invention will be pointed out in the appended claims. The apparatus of this invention is used with certain conventional components the details of which, although not filly illustrated or described, will be apparent to those having skill in the art and an understanding of the necessary function of such components.

More specifically an integrated olefin purification system including: one or more optional heat exchangers for controlling temperature of the gaseous feedstream to temperatures in a range downward from about 20° C. to about negative 35° C., adsorption vessels containing particulate beds of a suitable solid adsorbents, and means for analysis of feed and effluent streams, such as an on-line analytical system.

During operation of the integrated olefin purification system, a gaseous mixture containing less than about 500 parts per million by weight of the acetylene and carbon monoxide impurities formed by chemical conversions in commercial thermal cracking processes, is, for example ethylene fed from the overhead of a C2 distillation tower or intermediate storage through a feed exchanger to control temperature during adsorption. Effluent from feed exchanger flows into one of two adsorption vessels which contain beds of a suitable solid adsorbent, such as gamma alumina with 1.0 percent palladium based upon the weight of adsorbent.

During operation the gaseous mixture passes though the bed of particulate adsorbent at gas hourly space velocities in a range of from about 0.05 hours⁻¹ to about 20,000 hours⁻¹ and even higher, preferably from about 0.5 hours⁻¹ to about 10,000 hours⁻¹.

Compositions of the gaseous feed and effluent of each adsorption vessel is monitored by on-line analytical system. While levels of acetylenic impurities in the effluent of the adsorption vessel in purification service are in a range downward from a predetermined level, purified olefin from adsorption vessel flows directly to pipeline for transportation of polymer grade ethylene, or to storage. When the level of acetylenic impurities in the effluent of an adsorption vessel in purification service reaches or exceeds the predetermined level that adsorption vessel is isolated from the process flow and thereafter the resulting bed of loaded adsorbent is treated to effect release of the contained acetylenic impurities from the adsorbent by hydrogenation.

Suitable absorbents have capacity to treat from about 300 to about 40,000 pounds of olefin feed per pound of adsorbent where the olefin feed contains about 0.5 parts per million (ppm) acetylene. Approximately 5×10⁻⁴ pounds of acetylene to about 1×10⁻² pounds are, advantageously, adsorbed per pound of adsorbent before regeneration is required.

During continuous operation of this embodiment, the time required for treating, alternately, of the loaded adsorbent to effect release of the contained acetylenic impurities from the adsorbent by hydrogenation, is provided by using two or more independent adsorption vessels containing beds. Regenerations are, advantageously, performed according to this invention in three steps.

During the first stage of regeneration dry inert gas, such as methane, ethane, or nitrogen which is, preferably, free of carbon oxides, unsaturated hydrocarbons and hydrogen is fed, from, for example a nitrogen gas supply system exchanger to control temperature during regeneration. The dry inert gas flows through the bed of loaded adsorbent thereby purging gaseous hydrocarbons therefrom to disposal.

During the second stage of regeneration a reducing gas stream comprising dihydrogen, to effect release of the contained acetylenic impurities from the adsorbent. Preferably the reducing gas stream comprising predominately dihydrogen containing less than about 0.1 per million of carbon monoxide.

Where heating of the regeneration gas is desired, rates of temperature increase during the second stage of regeneration are, preferably, controlled to rates of less than about 11° C. per minute (about 20° F. per minute) while increasing temperature in the range of from about 4° C. to about 200° C. (about 40° F. to about 400° F.). Pressures of the hydrogen-rich reducing gas during the second stage of regeneration are, advantageously, in a range from about 5 psig to about 500 psig. While the reducing gas is flowing through the adsorbent bed, effluent gas composition is, periodically, monitored with gas analyzer. Second stage of regeneration is complete when C2+ hydrocarbon levels in the effluent gas from the bed have been reduced to C2+ hydrocarbon levels in the feed.

Third stage regeneration involves purging all gaseous hydrogen from the adsorption vessel with an inert gas, e.g. nitrogen with or without a saturated hydrocarbon gas such as methane or ethane, while the vessel is at temperatures in a range upward from about 60° C. (140° F.). During this third stage of regeneration flow of inert gas, at or below ambient temperature and about 5 to about 100 psig, cools the vessel to about ambient temperature thereby completing the regeneration process.

Surface area of adsorbents can be determined by the Brunaur-Emmett-Teller (BET) method or estimated by a simpler Point B method. Adsorption data for nitrogen at the liquid nitrogen temperature, 77 K, are usually used in both methods. The Brunaur-Emmett-Teller equation, which is well known in the art, is used to calculate the amount of nitrogen for mono-layer coverage. The surface area is taken as the area for mono-layer coverage based on the nitrogen molecular area, 16.2 square Angstroms, obtained by assuming liquid density and hexagonal close packing. In the Point B method, the initial point of the straight portion of the Type II isotherm is taken as the completion point for the mono-layer. The corresponding amount adsorbed multiplied by molecular area yields the surface area.

Dispersion and surface area of active metal sites was determined by carbon monoxide chemisorption using a Pulse Chemisorb 2700 (Micromeritics). In this procedure, approximately 4 gram samples were purged with helium carrier gas, calcined in air at 500° C. for 1 hr, purged with helium, reduced in hydrogen at 500° C., purged with helium, and cooled to room temperature. The sample was treated with 49.5 percent carbon monoxide in helium and the dosed with 0.045 mL pulses of 49.5 percent carbon monoxide (CO), balance nitrogen, and the carbon monoxide uptake was measured by a thermal conductivity cell. Palladium dispersion values were calculated assuming one carbon monoxide molecule per palladium atom. Palladium loadings are weight percent palladium metal.

In characterizing the pore volume, both total pore volume and its distribution over the pore diameter are needed. The total pore volume is usually determined by helium and mercury densities or displacements. Helium, because of its small atomic size and negligible adsorption, gives the total voids, whereas mercury does not penetrate into the pores at ambient pressure and gives inter-particle voids. The total pore volume equals the difference between the two voids.

Palladium on a high-surface-area γ-Al₂O₃ is a preferred adsorbent for purification of olefins in accordance with this invention. In order to introduce palladium and/or other suitable metal ions on a high-surface-area γ-Al₂O₃, any known technique for monolayer dispersion can be employed. The phenomenon of spontaneous dispersion of metal oxides and salts in monolayer or submonolayer forms onto surfaces of inorganic supports with high surface areas has been studied extensively in the literature (e.g., Xie and Tang, 1990).

### EXAMPLES OF THE INVENTION

The following Examples will serve to illustrate certain specific embodiments of the herein disclosed invention. These Examples should not, however, be construed as limiting the scope of the novel invention as there are many variations which may be made thereon without departing from the spirit of the disclosed invention, as those of skill in the art will recognize.

### Example 1

This example illustrates the procedure used to initially reduce a commercially available adsorbent, and then demonstrates use of an adsorption bed operating at 49° C. after the bed was regenerated with a reducing gas comprising dihydrogen and essentially free of carbon monoxide. It is noted that no carbon monoxide was present in the effluent gas from this adsorption bed.

Initial Reduction of Pd/γ-Al₂O₃ Adsorbent.

A 50 mL TEFLON-lined stainless steel pressure vessel was loaded with 18.88 gm of commercially available adsorbent (about 41 mL of 0.36 percent palladium on γ-Al₂O₃), and a centrally disposed thermocouple system to monitor bed temperatures. After this adsorption vessel was connected into a gas adsorption unit which provided required control of feed gases, temperatures, pressures and analytical means, the adsorbent bed was run in the down-flow mode. Nitrogen was purged through the vessel before reducing the oxidized PdO/γ-Al₂O₃ adsorbent by heating to 42° C. in a flow of hydrogen. A circulating water bath was used to supply heated needed during reduction at 80 psig with hydrogen flowrates of around 250 mL/min. After 16 hours hydrogen flow was replaced with nitrogen flow. The vessel was maintained at a temperature of about 49° C. during the subsequent adsorption process.

First Period of Adsorption for Pd/γ-Al₂O₃ Adsorbent.

After analysis of the effluent gases showed that hydrogen had been purged from the vessel, a feed mixture which contained 208 ppm acetylene in balance of ethylene was introduced into the vessel and the vessel was pressurized to 200 psig. During adsorption the flow rate of the acetylene/ethylene mixture was 201 mL/min. and operating conditions of pressure and temperature were controlled at 200 psig and 49° C. By periodical analysis of effluent gas using an on-line gas chromatograph, acetylene was detected (less than about 0.5 ppm acetylene) breaking through the bed of adsorbent after a total of about 103 L (1 atm and 21° C.) of feed gas was treated. In this first period of adsorption, the adsorbent exhibited a capacity of about 0.31 mL of acetylene per mL of adsorbent.

Initial Regeneration of Pd/γ-Al₂O₃ Adsorbent.

After flow of the acetylene/hydrogen/ethylene mixture was stopped, the vessel was depressured to 1 atm and nitrogen was purged through the vessel for about 15 minutes. The vessel was maintained at 49° C. using the circulating water bath. The adsorbent was regenerated using with a reducing gas comprising dihydrogen and essentially free of carbon monoxide at a flow rate of 250 mL/min. at 80 psig for about 16 hours. After regeneration for this period, hydrogen flow was replaced with nitrogen flow.

Second Period of Adsorption for Pd/γ-Al₂O₃ Adsorbent.

After analysis of the effluent gases showed that hydrogen had been purged from the vessel, a feed mixture which contained 208 ppm acetylene in balance ethylene was introduced into the vessel and the vessel was pressurized to 200 psig. During adsorption the flow rate of the acetylene/ethylene mixture was 202 mL/min. and operating conditions of pressure and temperature were controlled at 200 psig and 49° C. Periodical analysis of the effluent gas was made using an on-line gas chromatograph. No acetylene was detected in the effluent from the adsorbent bed until a total of 96 L (1 atm and 21° C.) of feed gas had been treated. No CO was detected in the effluent from the adsorbent bed at any time. In this example the adsorbent exhibited a capacity of about 0.23 mL of acetylene per mL of adsorbent.

### Examples 2-4

The regenerated adsorbent from Example 1 was subjected to three more adsorption and regeneration cycles using the methodology described in Example 1. The adsorption capacities of these three replicate runs were 0.28, 0.28, and 0.29 mL of acetylene per mL of adsorbent, respectively. Therefore, for adsorption at 49° C. and regeneration with pure hydrogen gas, the average adsorption capacity was 0.270 mL of acetylene per mL of adsorbent, with a standard deviation of 0.027 mL of acetylene per mL of adsorbent.

### Example 5

This example demonstrates use of an adsorption bed operating at 49° C. after the bed was regenerated with a reducing gas comprising dihydrogen and about 300 ppm by volume of carbon monoxide. It clearly shows that when carbon monoxide is present in the regeneration gas, carbon monoxide is also detected in the effluent gas from the adsorption bed during the subsequent adsorption cycle.

Flow of the acetylene/ethylene mixture to the spent adsorbent bed from Example 4 was stopped, the vessel was depressured to 1 atm and nitrogen was purged through the vessel for about 15 minutes. The vessel was maintained at 49° C. using the circulating water bath. The adsorbent was regenerated using a gas containing 21.1 mol percent dihydrogen, 282 ppm by volume of carbon monoxide, 4.77 mol percent ethylene (C₂H₄), in balance methane at a flow rate of 250 mL/min. at 80 psig for about 17 hours. After regeneration for this period, regeneration gas flow was replaced with nitrogen flow.

After analysis of the effluent gases showed that the regeneration gas had been purged from the vessel, a feed mixture which contained 180 ppm acetylene in balance ethylene was introduced into the vessel and the vessel was pressurized to 200 psig. During adsorption the flow rate of the acetylene/ethylene mixture was 202 mL/min. and operating conditions of pressure and temperature were controlled at 200 psig and 49° C. By periodical analysis of effluent gas using an on-line gas chromatograph, acetylene was detected (less than about 0.5 ppm acetylene) breaking through the bed of adsorbent after a total of 159 L (1 atm and 21° C.) of feed gas was treated. In this example the adsorbent exhibited a capacity of about 0.38 mL of acetylene per mL of adsorbent. The initial gas chromatograph of the adsorbent bed effluent showed 134 ppm of carbon monoxide. All subsequent analyses showed between 15 to 30 ppm of carbon monoxide in the effluent of the adsorption bed.

### Examples 6-9

The spent adsorbent from Example 5 was subjected to four more regeneration and adsorption cycles using the methodology described in Example 5. The adsorption capacities of these four replicate runs were 0.28, 0.31, 0.39, and 0.41 mL of acetylene per mL of adsorbent, respectively. Therefore, for adsorption at 49° C. and regeneration with CO-containing gas, the average adsorption capacity was 0.354 mL of acetylene per mL of adsorbent, with a standard deviation of 0.056 mL of acetylene per mL of adsorbent.

### Example 10

This example of the invention demonstrates the operation of an adsorption bed at negative 31° C. after being regenerated with a pure hydrogen gas. Flow of the acetylene/ethylene mixture to the spent adsorbent bed from Example 9 was stopped, the vessel was depressured to 1 atm and nitrogen was purged through the vessel for about 15 minutes. The temperature of the vessel was held at 49° C. using a circulating water bath. The adsorbent was regenerated using a pure dihydrogen gas at 250 mL/min. and 80 psig for about 17 hours. After regeneration for this period, hydrogen flow was replaced with nitrogen flow.

After analysis of the effluent gases showed that the regeneration gas had been purged from the vessel, the vessel was placed in a cooling bath filled with isopropanol and chilled to −31° C. A feed mixture which contained 185 ppm acetylene in balance ethylene was introduced into the vessel and the vessel was pressurized to 200 psig. During adsorption the flow rate of the acetylene/ethylene mixture was 112 mL/min. and operating conditions of pressure and temperature were controlled at 425 psig and negative 31° C. By periodical analysis of effluent gas using an on-line gas chromatograph, acetylene was detected (less than about 0.5 ppm acetylene) breaking through the bed of adsorbent after a total of 109 L (1 atm and 21° C.) of feed gas was treated. In this example the adsorbent exhibited a capacity of about 0.26 mL of acetylene per mL of adsorbent.

### Example 11

The spent adsorbent from Example 10 was subjected to one more regeneration and adsorption cycle using the methodology described in Example 10. The adsorption capacity of this replicate run was 0.25 mL of acetylene per mL of adsorbent. Therefore, for adsorption at negative 31° C. and regeneration with pure hydrogen gas, the average adsorption capacity was 0.255 mL of acetylene per mL of adsorbent, with a standard deviation of 0.007 mL of acetylene per mL of adsorbent.

### Example 12

This example of the invention demonstrates the operation of an adsorption bed at negative 31° C. after being regenerated with a gas containing both dihydrogen and carbon monoxide. Flow of the acetylene/ethylene mixture to the spent adsorbent bed from Example 11 was stopped, the vessel was depressured to 1 atm and nitrogen was purged through the vessel for about 15 minutes. The vessel temperature was held at 49° C. using a circulating water bath. The adsorbent was regenerated using a gas containing 21.1 mol percent dihydrogen, 282 ppm by volume of carbon monoxide, 4.77 mol percent ethylene (C₂H₄), in balance methane at a flow rate of 250 mL/min. at 80 psig for about 16 hours. After regeneration for this period, regeneration gas flow was replaced with nitrogen flow.

After analysis of the effluent gases showed that the regeneration gas had been purged from the vessel, the vessel was placed in a cooling bath filled with isopropanol and chilled to negative 31° C. A feed mixture which contained 180 ppm acetylene in balance ethylene was introduced into the vessel and the vessel was pressurized to 200 psig. During adsorption the flow rate of the acetylene/ethylene mixture was 200 mL/min. and operating conditions of pressure and temperature were controlled at 200 psig and −31° C. By periodical analysis of effluent gas using an on-line gas chromatograph, acetylene was detected (less than about 0.5 ppm acetylene) breaking through the bed of adsorbent after a total of 53 L (1 atm and 21° C.) of feed gas was treated. In this example the adsorbent exhibited a capacity of about 0.127 mL of acetylene per mL of adsorbent.

### Examples 13-17

The spent adsorbent from Example 12 was subjected to five more regeneration and adsorption cycles using the methodology described in Example 12. The adsorption capacities of these five replicate runs were 0.12, 0.09, 0.07, 0.18, and 0.18 mL of acetylene per mL of adsorbent, respectively. Therefore, for adsorption at negative 31° C. and regeneration with CO-containing gas, the average adsorption capacity was 0.128 mL of acetylene per mL of adsorbent, with a standard deviation of 0.045 mL of acetylene per mL of adsorbent.

Data of these examples clearly demonstrate that regeneration of the loaded bed of adsorbent in the presence of a reducing gas comprising dihydrogen which is free of a substantial amount of carbon monoxide produced a regenerated bed having high capacities for acetylenic impurities. In particular, the average adsorption capacities were 0.270 mL of acetylene per mL of adsorbent and 0.255 mL of acetylene per mL of adsorbent where the adsorption was carried out respectively at 49° C. and at negative 31° C. By contrast, when the adsorption was carried out at negative 31° C. and the regeneration of the loaded bed of adsorbent was carried out in the presence of a reducing gas comprising dihydrogen containing of a substantial amount of carbon monoxide the average, the resulting average adsorption capacity was only 0.128 mL of acetylene per mL of adsorbent. where

For the purposes of the present invention, “predominantly” is defined as more than about ninety per cent. “Substantially” is defined as occurring with sufficient frequency or being present in such proportions as to measurably affect macroscopic properties of an associated compound or system. Where the frequency or proportion for such impact is not clear substantially is to be regarded as about twenty per cent or more. The term “Essentially” is defined as absolutely except that small variations which have no more than a negligible effect on macroscopic qualities and final outcome are permitted, typically up to about one percent.

Examples have been presented and hypotheses advanced herein in order to better communicate certain facets of the invention. The scope of the invention is determined solely by the scope of the appended claims.

That which is claimed is:

1. A process for purification of an olefin stream to obtain a feedstock suitable for formation of olefin polymers, which purification process comprises:
providing an impure gaseous mixture comprising at least one olefin of from 2 to about 8 carbon atoms, acetylenic impurities having the same or similar carbon content in an amount of up to about 1 percent by volume based upon the total amount of olefin present and optionally saturated hydrocarbon gases;
passing the impure mixture through a bed of regenerated adsorbent which is free of a substantial amount of carbon monoxide, the adsorbent comprising predominantly a support material having high surface area on which is dispersed at least one metallic element in the zero valent state selected from the group consisting of chromium, iron, cobalt, nickel, copper, ruthenium, palladium, silver and platinum, to effect, under conditions suitable for adsorption within the bed, selective adsorption and/or complexing of the contained acetylenic contaminants with the adsorbent, and thereby obtain an effluent mixture which contains less than about 1 part per million by volume of the acetylenic impurities and an amount of carbon monoxide less than is deleterious to formation of olefin polymers; and
thereafter regenerating the resulting bed of adsorbent in the presence of a reducing gas comprising dihydrogen which is free of a substantial amount of carbon monoxide, to effect release of the contained acetylenic impurities from the adsorbent.

2. The process according to claim 1 wherein the gaseous mixture, while passing through the bed, is at temperatures in a range downward from about 20° C. to about negative 35° C.

3. The process according to claim 1 wherein the reducing gas comprises at least 25 percent dihydrogen, less than 0.5 parts per million carbon monoxide, and optionally saturated hydrocarbon and/or inert gases.

4. The process according to claim 1 wherein the support is a material selected from the group consisting of alumina, silica, active carbon, clay and zeolites, and has surface area in a range of from about 10 to about 2,000 square meters per gram as measured by the BET gas adsorption method.

5. The process according to claim 1 wherein the gaseous mixtures pass though the beds of adsorbent at gas hourly space velocities in a range of from about 0.05 hours⁻¹ to about 20,000 hours⁻¹ measured at standard conditions of 0° C. and 760 mm Hg.

6. The process according to claim 1 wherein the adsorbent comprises at least about 90 weight percent of a gamma alumina having surface area in a range of from about 80 to about 500 square meters per gram as measured by the BET gas adsorption method, and contains less than 500 parts per million by weight of a sulfur-containing component, calculated as elemental sulfur.

7. The process according to claim 6 wherein the metal dispersed on the support material is palladium, and the absorbent has a palladium content in a range of from about 0.01 to about 10 percent based on the total weight of the adsorbent.

8. The process according to claim 1 wherein the olefin in the gaseous mixture being purified is predominantly ethylene or propylene, the gaseous mixture contains less than about 0.5 parts per million by volume of dihydrogen and less than about 1 parts per million by volume of mercury-containing, arsenic-containing, and sulfur-containing components.

9. The process according to claim 8 wherein the adsorbent comprises at least about 90 weight percent of a gamma alumina having surface area in a range of from about 150 to about 350 square meters per gram as measured by the BET gas adsorption method, and wherein the metal dispersed on the support material is palladium, and the absorbent has a palladium content in a range of from about 0.01 to about 10 percent based on the total weight of the adsorbent.

10. The process according to claim 9 wherein the gaseous mixture, while passing through the bed, is at temperatures in a range downward from about 20° C. to about minus 35° C.

11. A process for purification of olefins produced by thermal cracking of hydrocarbons to obtain a feedstock suitable for formation of olefin polymers, which purification process comprises:
providing an impure gaseous mixture comprising at least about 99 percent by volume of an olefin having from 2 to about 4 carbon atoms, and acetylenic impurities having the same or similar carbon content in an amount in a range upward from about 1 to about 1000 parts per million by volume based upon the total amount of olefin present and optionally saturated hydrocarbon gases;
passing the impure mixture through a bed of regenerated adsorbent which is free of a substantial amount of carbon monoxide, the adsorbent comprising predominantly a support material selected from the group alumina, silica, active carbon, clay and zeolites having surface area in a range of from about 10 to about 2,000 square meters per gram as measured by the BET gas adsorption method, on which is dispersed at least one metallic element in the zero valent state selected from the group consisting of chromium, iron, cobalt, nickel, copper, ruthenium, palladium, silver and platinum, to effect, under conditions suitable for adsorption within the bed, selective adsorption and/or complexing of the contained acetylenic contaminants with the adsorbent;
effecting, in the presence of an essentially dihydrogen-free atmosphere within the bed, selective adsorption and/or complexing of the contained acetylenic impurities with the adsorbent, until levels of the acetylenic impurities in the effluent mixture increase to a limiting level in a range downward from about 1 parts per million by volume; and
thereafter regenerating the resulting bed of adsorbent in the presence of a reducing gas comprising dihydrogen which reducing gas is free of a substantial amount of carbon monoxide, to effect release of the contained acetylenic impurities from the adsorbent.

12. The process according to claim 11 wherein the gaseous mixture, while passing through the bed, is at temperatures in a range downward from about 5° C. to about negative 35° C.

13. The process according to claim 11 wherein the reducing gas comprises less than 0.1 parts per million carbon monoxide, and at least 90 percent dihydrogen.

14. The process according to claim 11 wherein the adsorbent has a metal dispersion value in a range upward from about 20 percent to about 80 percent as measured by carbon monoxide chemisorption method.

15. The process according to claim 11 wherein the gaseous mixture passes though the bed of particulate adsorbent at gas hourly space velocities in a range of from about 0.05 hours⁻¹ to about 20,000 hours⁻¹ measured at standard conditions of 0° C. and 760 mm Hg.

16. The process according to claim 11 wherein the adsorbent comprises at least about 90 weight percent of a gamma alumina having surface area in a range of from about 80 to about 500 square meters per gram as measured by the BET gas adsorption method, and contains less than 500 parts per million by weight of a sulfur-containing component, calculated as elemental sulfur.

17. The process according to claim 16 wherein the metal dispersed on the support material is palladium, and the absorbent has a palladium content in a range of from about 0.01 to about 10 percent based on the total weight of the adsorbent.

18. The process according to claim 17 wherein the olefin in the gaseous mixture being purified is predominantly ethylene or propylene, the gaseous mixture contains less than about 0.5 parts per million by volume of hydrogen and less than about 1 parts per million by volume of mercury-containing, arsenic-containing, and sulfur-containing components, each calculated as the element, and wherein the gaseous mixture, while passing through the bed, is at temperatures in a range of from about negative 35° C. to about 5° C.

19. A process for purification of a predominantly ethylene stream to obtain a feedstock suitable for formation of polymer, which purification process comprises:
providing an impure gaseous stream comprising at least about 95 percent by volume of ethylene, and acetylene in an amount in a range upward from about 1 to about 1000 parts per million by volume based upon the total amount of ethylene present and optionally saturated hydrocarbon gases;
passing the impure stream through a bed of adsorbent which is free of a substantial amount of carbon monoxide, the adsorbent comprising at least about 90 weight percent of gamma alumina having surface area in a range of from about 150 to about 350 square meters per gram as measured by the BET gas adsorption method, on which is dispersed is at least one element selected from the group consisting of iron, cobalt, nickel, copper, palladium, silver and platinum, in the zero valent state, to effect, under conditions suitable for adsorption within the bed, selective adsorption and/or complexing of the contained acetylene contaminant with the adsorbent, thereby obtain an effluent steam of feedstock which contains less than about 0.5 parts per million by volume of carbon monoxide and less than about 1 parts per million by volume of the acetylene and;
effecting, in the presence of an essentially dihydrogen-free atmosphere within the bed, selective adsorption and/or complexing of the contained acetylene with the adsorbent, until levels of the acetylene in the effluent stream increase to a limiting level in a range downward from about 1 parts per million by volume; and
thereafter regenerating the resulting bed of adsorbent in the presence of a reducing gas comprising dihydrogen which reducing gas is free of a substantial amount of carbon monoxide, to effect release of the contained acetylene from the adsorbent.

20. The process according to claim 19 wherein the adsorbent comprises at least about 90 weight percent of a gamma alumina having surface area in a range of from about 150 to about 350 square meters per gram as measured by the BET gas adsorption method, the metal dispersed on the support material is palladium, and the absorbent has a palladium content in a range of from about 0.01 to about 10 percent based on the total weight of the adsorbent, and wherein the gaseous mixture, while passing through the bed, is at temperatures in a range of from about negative 35° C. to about 5° C.
